(19) United States
(12) Patent Application Publication
(10) Pub. No.: US 2009/0038625 A1
(43) Pub. Date: Feb. 12, 2009

(54) Earplug with shell and insertion stem

(76) Inventors: Raul Cortez, Chula Vista, CA (US); Stephen D. Gilder, Chula Vista, CA (US)

Correspondence Address: LEON D. ROSEN;FREILICH, HORNBAKER & ROSEN, Suite 1220, 10960 Wilshire Blvd.,, Los Angeles, CA, 90024, US

(21) Appl. No.: 11/891,126

(22) Filed: Aug. 9, 2007

(51) Int. Cl. A61F 11/08

(52) U.S. Cl. 128/864

(57) ABSTRACT

An earplug of the type that includes a shell (12) that fits tightly in a human ear canal and a stem (20) that lies in the shell and can be pushed forward to push the shell into an ear canal, which reduces the possibility of loss of the stem, and provides better blocking of noise. The shell and stem have front ends that are integrally molded and that merge to avoid loss of the stem, although the molding leaves gaps between the shell and stem. The stem has a passage (62), and an insert (60) is inserted into the passage and expands the stem to cause the stem to closely slidably fit in a guide flange (44) of the shell and to press the stem into contact with an inside flange (46) of the shell to form a seal thereat to better block sound. An additional inside front flange (62) is formed in a front portion of the shell, so when the shell front portion is compressed by the walls of the ear canal the front flange engages the stem to provide another sealed chamber that blocks sound.

### BACKGROUND OF THE INVENTION

One type of earplug, described in U.S. Pat. Nos. 4,434,794 and 6,006,857, includes a shell of elastomeric material and a separate stem that is inserted into the shell to extend though it and lie against the front end of the inside of the shell. To insert the front portion of the shell into a person's ear canal, the stem is pushed forward to push the front end of the shell forward. The shell temporarily elongates and decreases slightly in diameter, which facilitates such insertion. The stem may pull out of the shell especially if the earplug is removed from the ear by pulling on the rear end of the stem. An earplug of the type that includes an elastomeric shell and a stem that can be pushed forward to install the shell in a person's ear canal, which prevented separation of the stem from the shell, would be of value.

### SUMMARY OF THE INVENTION

In accordance with one embodiment of the invention, an earplug is provided of the type that includes an elastomeric shell and a stem that lies in the shell and that can be pushed forward to install the shell in a person's ear canal, which prevents separation of the shell from the stem. The stem and shell are molded at the same time, with their front ends molded integrally with each other. This prevents the shell and stem from separating, other than by tearing them apart. To mold the shell and stem integrally, a core is used that lies in the space between them. As a result, internal flanges in the shell do not initially contact the stem. Contact between a shell internal flange and the shell is important to block sound, and a close sliding fit between a shell internal flange and the shell is useful to guide sliding of the stem. Such contact and close sliding fit are achieved by leaving a passage in the stem and inserting an insert into the passage. The insert expands location on the stem into contact with a flange or into a close sliding fit of the stem with a guiding flange on the inside of the shell.

An inside flange can be provided in a front portion of the shell, with the shell front portion compressed by the ear canal into engagement with the stem.

The novel features of the invention are set forth with particularity in the appended claims. The invention will be best understood from the following description when read in conjunction with the accompanying drawings.

### BRIEF DESCRIPTION OF THE DRAWINGS

FIG. 1 is a sectional view of an earplug of the present invention, as it is molded, the earplug being symmetric about its axis.

FIG. 2 is a sectional view of the earplug of FIG. 1, after an expander is inserted into a passage of a stem of the earplug.

FIG. 3 is a sectional view of the earplug of FIG. 2, after the front portion of the earplug is inserted into the ear canal of a person.

### DESCRIPTION OF THE PREFERRED EMBODIMENTS

FIG. 1 illustrates an earplug 10 of the invention, which includes a shell 12 with a shell chamber 14 extending along most of the length of the shell to a shell front end 16, and with a shell inner surface 18. The earplug also includes a stem 20 that extends along most of the length of the shell, and that has a stem front end 22. The shell front end 16 and the stem front end 22 are molded integrally, so they merge into a shell-stem combination 19. The only way to separate the shell from the stem is to tear them apart.

The shell and stem are molded of soft and preferably elastomeric material. The shell has a front portion 24 that is designed to lie in a person's ear canal to block sound. The shell also has a rear portion 26 with a rear part 30 that is flared so it is of progressively greater diameter in a rearward direction R, and that is intended to lie primarily outside the ear canal. The average adult ear canal has a diameter of about 0.30 inch (most are in the range of about 0.27 inch to 0.33 inch). The uncompressed earplug front portion 24 has a maximum outside diameter A of about 0.42 inch at a shell outside location 32 which has the largest diameter of the earplug front portion 24. In FIG. 1, a shell front portion 36 extends from the earplug extreme front end 34, and rearwardly along a length B equal to the maximum uncompressed diameter A of the shell front portion that fits tightly in the ear canal.

To install the earplug in a person's ear canal, a person pushes the rear of the stem forward F along the earplug axis 40. The stem slides slightly within the shell inner surface 18, and is guided in such sliding by a guide 42 formed by a radially inwardly (i.e. toward axis 40) projecting second shell flange 44 that should lie closely around the stem in a sliding fit. A first inward flange 46 is provided that should contact the stem, to stop the passage of sound to the front of the shell inside surface through thin shell front walls 50 into the person's ear canal. When the earplug is initially molded, a molding core in the shape of chamber 14 occupies the space between the shell and stem, so there is a gap at 52 and 54 inside the first and second flanges 46, 44 and the outside surface of the stem. The gaps require a minimum thickness on the order of magnitude of 3 millimeters, so the core will not break during withdrawal.

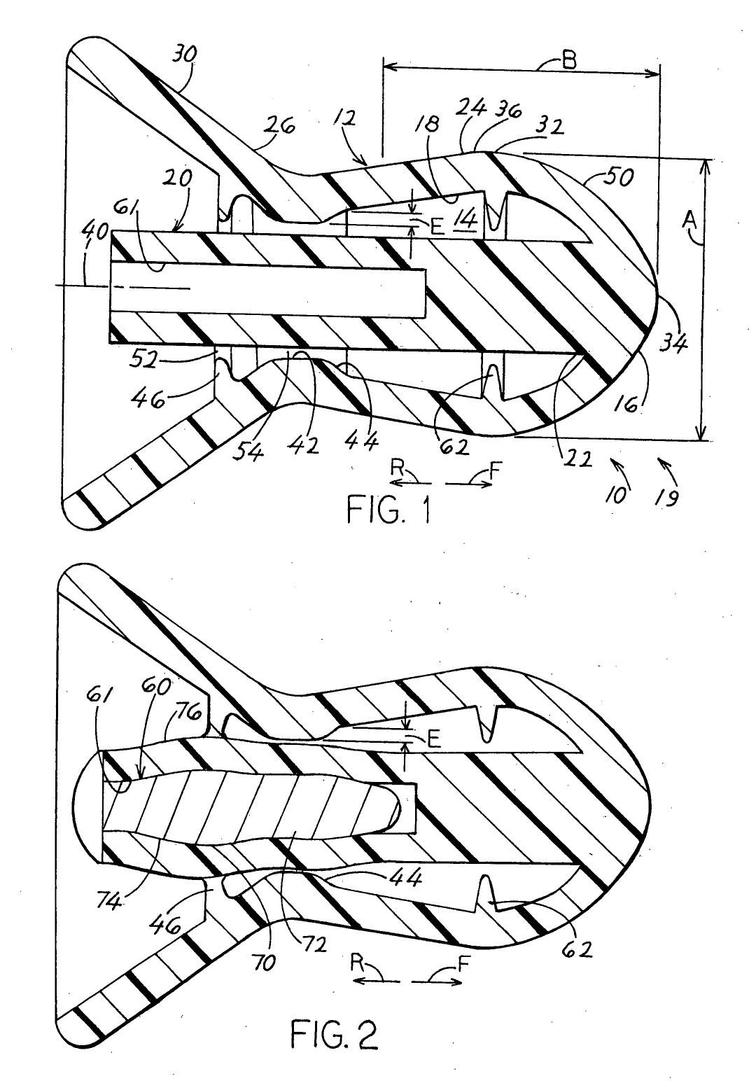

FIG. 2 shows the earplug of FIG. 1 after an insert 60 has been installed in the stem passage 62. The insert is of stiffer material than that of the stem, and helps stiffen the stem to prevent stem column collapse during earplug insertion. In addition, the insert has enlarged parts such as 70 and 72 that expand the stem and cause the stem outside surface 76 to expand in diameter at locations inside the first and second internal shell flanges 46, 44. The insert expands the stem at a first location that lies inside the first flange 46, and expands the stem first location sufficiently that the stem outside surface 76 contacts the first flange 46 and preferably forms a slight interference fit with the first flange 46. The insert expands the stem at a second location inside the second flange 44 so the second flange lies closer to the stem but in a sliding fit therewith.

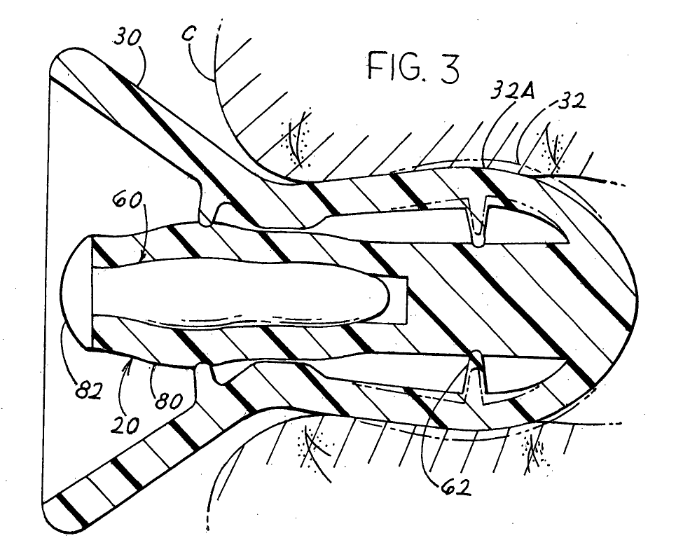

FIG. 3 shows the earplug of FIG. 2, which includes the insert 60, after the earplug has been installed in the ear canal C of a person and lies in an interference fit with the ear canal so shell location 32 has been compressed to 32A. The shell has a third, or front, internal flange 62. The front flange preferably lies directly inside the locations 32, 32A of the shell front location of maximum diameter. Initially, as in FIGS. 1 and 2, the front flange 62 lies out of contact with the stem 20. However, when the earplug has been inserted into the ear canal, the ear canal compresses the shell location 32. The compression is sufficient to cause the front internal flange 62 to move into contact, and preferably into a slight interference fit, with the outside of the stem 20.

A person can remove the earplug from the ear canal by grasping the rear part 30 of the shell or grasping the rear end portion 80 of the stem and pulling out the earplug. The insert 60 cannot be readily grasped, so a person does not grasp it to pull out the earplug. The insert has a rear end 82 that is normally pushed forward during installation of the earplug into the ear canal. However, the insert rear end 82 is very difficult to grasp, so a person will not grasp it to pull the earplug out of an ear canal.

The combination of the shell 12 and stem 20 (FIG. 1) are integrally molded of a soft elastomer (an elastomer has a Young's modulus of no more than 50,000 psi) such as one with a Shore A hardness of no more than 30. The particular shell and stem have a Shore A of 17. The insert 60 is preferably formed of material such as an engineering plastic (Young's modulus over 50,000) such as one with a shore A hardness of over 60.

Thus, the invention provides an earplug of the type that has a shell with a chamber therein, and a stem that lies in the chamber and that can be pressed forward to install the shell in a human ear canal. In the invention, the shell and stem are molded integrally, with the front ends of the shell and stem merging as a result of being integral. As a result, the shell and stem cannot be separated except by tearing them apart. When the earplug is molded, a core pin that forms the chamber that lies within the shell and outside the stem, leaves gaps between first and second shell internal flanges and the shell. The second flange 44 which guides the stem is sliding, is longer in front-rear longitudinal directions F, R than its radial projection E and is used to guide the stem in sliding, so it should lie close to the stem in a sliding fit therewith. The first flange 46 is intended to block sound, and preferably lies in contact with the stem outer surface. Applicant molds the earplug so the stem has a passage therein. Applicant provides an insert that expands the stem to lie in a close sliding fit with the second flange and in contact with the first stem, the insert also stiffening the stem. Applicant prefers to form the earplug so the shell has a front flange that lies within the largest diameter outside location of the shell front portion, which is the primary seal against the ear canal. The ear canal compresses the shell at the ear canal sealing location and thereby moves the front internal flange into contact with the stem.

Although particular embodiments of the invention have been described and illustrated herein, it is recognized that modifications and variations may readily occur to those skilled in the art, and consequently, it is intended that the claims be interpreted to cover such modifications and equivalents.

What is claimed is:

1. An earplug that includes a shell with a front portion that fits in an interference fit into a person's ear canal and a stem that fits in the shell and that has a stem rear end that can be urged forward to insert the shell into the ear canal, wherein:
said shell and stem each has a front end, and said shell and stem are integrally molded,with said shell and stem front ends merging.

2. The earplug described in claim 1 wherein:
said shell has a front portion with a location of maximum diameter;
said shell front portion has an internal flange that has an inside diameter that is initially greater than the outside diameter of the stem so said shell and flange are initially out of contact, but with the shell front portion being compressible by the person's ear canal when the earplug is inserted into the ear canal to radially inwardly deflect said internal flange into contact with said stem.

3. The earplug described in claim 1 wherein said shell has an axis and said stem is elongated along said axis, and wherein:
said shell has a shell front portion that fits in a person's ear canal, and a shell rear portion that has a rear end that is intended to lie outside the ear canal;
said shell having at least a first radially inwardly projecting flange at said shell rear portion;
said stem has a rear end and has a passage that extends to said passage rear end; and including
an insert that fits into said passage in said stem and that expands said stem sufficiently that said stem lies in contact with said first shell internal flange.

4. The earplug described in claim 3 wherein:
said shell has a pair of radially inwardly projecting internal flanges, including a guiding second flange, that are spaced apart along said axis;
said insert expands a first stem location lying directly within said first flange so said first stem location lies in contact with said first flange, and said insert expands a stem location lying directly within said second flange so the stem is expanded in diameter and said second flange slideably guides said stem in movement along said axis.

5. The earplug described in claim 1 wherein:
said shell has a front portion with a location of maximum outside diameter, and said shell has a front internal flange that lies within said location of maximum outside diameter, said front internal flange being of an initial diameter greater that a stem location lying within said front internal flange, and said front internal flange being radially inwardly deflected by a person's ear canal to engage the stem when said location of maximum outside diameter lies in a person's ear canal.

6. An earplug that includes a shell with an axis extending in front and rear directions, said shell having a front portion that fits into a person's ear canal to seal it and a rear portion, said earplug including a stem that lies in the shell, wherein:
said shell rear portion has a first internal flange that lies out of contact with said stem;
said stem has a stem passage extending along said axis, and including an insert that lies in an interference fit with walls of said stem passage and that expands a location of said stem into contact with said first internal flange.

7. The earplug described in claim 6 wherein:
said shell rear portion has a second internal flange; and
said expander is constructed to expand a location of said stem into a sliding fit with said second internal flange.

8. The earplug described in claim 7 wherein:
said first flange has a first radially inner end of a short length parallel to said axis, and said second flange has a radially inner end with a length (E) parallel to said axis that is at least twice said short length.

9. The earplug described in claim 6 wherein:
said shell front portion has a shell front end, and said stem has a front end that is molded integrally with said shell front end.

10. The earplug described in claim 6, wherein:
said shell and stem are integrally molded with front ends of said shell and stem merging;
said shell and stem are constructed of soft elastomeric material having a Shore A hardness of less than 30, and said insert is formed of material that is stiffer than the material of said shell and stem and that has a Shore A hardness of more than 60.

11. An earplug that includes a shell with a front portion that is insertable in an interference fit into a person's ear canal and a stem that lies in the shell and that has a stem rear end that can be urged forward to insert the shell into the ear canal, wherein:
said shell and stem each has a front end, and said shell and stem are integrally molded with said shell and stem front ends merging to form a shell-stem combination with gaps between said shell and stem immediately after molding;
said shell has at least one internal flange; and including
means for deflecting said combination to move locations on said flange and shell closer together.
